(19) United States
(12) Patent Application Publication
(10) Pub. No.: US 2010/0273838 A1
(43) Pub. Date: Oct. 28, 2010

(54) STABLE TOPICAL COMPOSITIONS FOR 1,2,4-THIADIAZOLE DERIVATIVES

(76) Inventors: Chengji Cui, Pennington, NJ (US); Shirley Mei-king NG, Bridgewater, NJ (US); George Wong, Belle Mead, NJ (US); Mohammed Yusuf, Hatfield, PA (US); Fa Zhang, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/765,981

(22) Filed: Apr. 23, 2010

Related U.S. Application Data
(60) Provisional application No. 61/172,526, filed on Apr. 24, 2009.

(51) Int. Cl. A61K 31/433; A61K 31/17; A61P 17/10

(52) U.S. Cl. 514/361; 514/586

(57) ABSTRACT

The present application provides a stable topical composition comprising a compound of 1,2,4-thiadiazole derivatives and the related thiourea derivatives. The stable topical composition may be present in various forms, including aqueous gel, cream, and emulsion. The stable topical composition may be stored at refrigerated or ambient condition for a reasonable shelf-life. The present application also provides a method of treating dermatologic disorders mediated by a melanocortin receptor using the stable topical composition. The stable composition may be delivered using a single chamber or dual chamber device. A method of preparing and delivering the stable composition is also provided.

### CROSS REFERENCE TO RELATED APPLICATIONS

This application claims the benefit of U.S. Provisional Application 61/172,526, filed on Apr. 24, 2009, which is incorporated by reference herein in its entirety.

### FIELD OF THE INVENTION

This invention relates to the topical delivery of 1,2,4-thiadiazole derivatives and the related thiourea derivatives in stable compositions suitable for the treatment of dermatologic disorders mediated by a melanocortin receptor.

### BACKGROUND OF THE INVENTION

1,2,4-thiadiazole derivatives and the related thiourea derivatives are useful for the treatment of a disorder mediated by a melanocortin receptor. U.S. Pat. Nos. 7,049,331 and 7,319,107 report that 1,2,4-thiadiazole derivatives are effective for the treatment of metabolic, CNS, and dermatologic disorders, such as obesity, impaired oral glucose tolerance, elevated blood glucose levels, type II diabetes, Syndrome X, diabetic retinopathy, acute neurodegenerative disorders, chronic neurodegenerative disorders, plexopathies, male erectile dysfunction, dry eyes, acne (e.g. acne vulgaris), dry skin, aged skin, seborrheic dermatitis, rosacea, excessive ear wax, meibomian gland disorder, pseudofolliculitis, yeast infections, dandruff, hiradenitis suppurativa, ocular rosacea and eccrine gland disorder. Both U.S. Pat. Nos. 7,049,331 and 7,319,107 are incorporated by reference herein in their entirety.

The members of 1,2,4-thiadiazole derivatives and the related thiourea derivatives bind to the melanocortin receptors such as melanocortin-5 (MC-5) receptor. U.S. Pat. Nos. 7,049,331 and 7,319,107 disclose topical formulations comprising these compounds. However, these formulations may not be the optimum formulations for commercialization. For example, one of the formulations disclosed in these patents was a hydroalcoholic gel with a pH of between 3 to 4, which is more acidic than is desirable for a topical formulation. It is preferable for topical formulations to have a pH that is slightly acidic e.g. pH 5 to 6.

Topical delivery of active pharmaceutical ingredients for the treatment of dermatological diseases requires carriers or compositions compatible with pharmaceutical active ingredients and suitable for the desired shelf-life and intended disease treatment. In general, an anhydrous carrier such as petrolatum or oil vehicle may be used for dissolving or solubilizing hydrophobic pharmaceutical active ingredients. However, the occlusive property of these hydrophobic vehicles and their inability to release the pharmaceutical active ingredients may reduce the efficacy of the composition for topical administration. In addition, the occlusive properties of these vehicles may not be suitable for treatment of certain dermatological disease such as acne. Alternatively, a water-based carrier in combination with additional agent such as a solubilizer may be used for hydrophobic pharmaceuticals. The solubilizer may increase the solubility of the hydrophobic ingredients in an aqueous environment and may affect the efficacy of the pharmaceutical ingredients for topical administration. Therefore, careful consideration of the carriers is necessary to create an efficacious pharmaceutical formulation with a desirable shelf-life.

Thus, it is an object of the present invention to provide stable pharmaceutical compositions or carriers with enhanced properties for this new class of 1,2,4-thiadiazole derivatives and the thiourea derivatives in a manner suitable for topical administration onto the skin for treating dermatological diseases such as acne or extensive sebum production.

### SUMMARY OF THE INVENTION

In one aspect, the invention features a composition containing a compound of Formula I, II, or III (defined herein) in an amount of about 0.05% to about 20% by weight, a viscosity modifying agent in an amount of about 0.1% to about 20% by weight, a preservative in an amount of about 0.05% to about 5% by weight, optionally a pH modifying agent in an amount of about 0.05% to about 0.5% by weight, and water added in an amount quantum sufficiat to provide a total of 100% by weight, wherein the compound of Formula I, II, or III in the composition is stable.

In another aspect, the invention features a method of preparing a topical composition comprising mixing (a) an aqueous gel comprising a compound of Formula I, II, or III in an amount of about 0.1% to about 20% by weight, a viscosity modifying agent in an amount of about 0.1% to about 20% by weight, a preservative in an amount of about 0.05% to about 5% by weight, optionally a pH modifying agent in an amount of about 0.05% to about 0.5% by weight, and water in an amount quantum sufficiat to provide a total of 100% by weight; with (b) a base comprising a mixture of solvents in an amount of about 1% to about 20% by weight, a viscosity modifying agent in an amount of about 0.5% to about 20% by weight, a preservative in an amount of about 0.05% to about 5% by weight; an emulsifier in an amount of about 0.2% to about 10% by weight, a pH modifying agent in an amount of about 0.05% to about 0.5% by weight, and water in an amount quantum sufficiat to provide a total of 100% by weight; to form a substantially homogeneous topical composition.

In another aspect, the invention features a method of treating a dermatological disease or disorder mediated by a melanocortin receptor in a subject in need thereof comprising administering an effective amount of the composition according to the present invention.

In yet another aspect, the invention features a kit comprising two chambers, wherein the first chamber contains an aqueous gel comprising a compound of Formula I, II, or III in an amount of about 0.1% to about 20% by weight, a viscosity modifying agent in an amount of about 0.1% to about 20% by weight, a preservative in an amount of about 0.05% to about 5% by weight, optionally a pH modifying agent in an amount of about 0.05% to about 0.5% by weight, and water in an amount quantum sufficiat to provide a total of 100% by weight; and the second chamber contains a base comprising a mixture of solvents in an amount of about 1% to about 20% by weight, a viscosity modifying agent in an amount of about 0.5% to about 20% by weight, a preservative in an amount of about 0.05% to about 5% by weight; an emulsifier in an amount of about 0.2% to about 10% by weight, a pH modifying agent in an amount of about 0.05% to about 0.5% by weight, and water in an amount quantum sufficiat to 100% by weight. A kit, depending on its design, is suitable for unit dose or multiple dose dispensing.

Other features and advantages of the present invention will be apparent from the detailed description of the invention and from the claims.

### DETAILED DESCRIPTION OF THE PREFERRED EMBODIMENTS

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

The present invention relates to a stable topical composition comprising a compound of 1,2,4-thiadiazole derivatives and the related thiourea derivatives. The structure of 1,2,4-thiadiazole derivates is provided below.

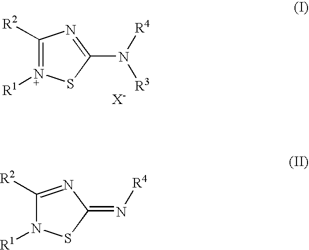

wherein:

R1 is selected from the group consisting of aryl, aralkyl, heteroaryl, heteroaryl-alkyl, heterocycloalkyl, heterocycloalkyl-alkyl, cycloalkyl and cycloalkyl-alkyl; wherein the aryl, aralkyl, heteroaryl, heterocycloalkyl, heterocycloalkyl-alkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;

R2 is selected from the group consisting of aryl, aralkyl, heteroaryl, heterocycloalkyl and cycloalkyl-alkyl; wherein the aryl, aralkyl, heteroaryl, heterocycloalkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;

R3 is selected from the group consisting of hydrogen, alkyl, alkenyl and alkynyl; wherein the double bond of the alkenyl or the triple bond of the alkynyl group is at least one carbon atom removed from the point of attachment;

R4 is selected from the group consisting of aryl, aralkyl, heteroaryl, heterocycloalkyl, and cycloalkyl-alkyl; wherein the aryl, aralkyl, heteroaryl, heterocycloalkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;

X— is selected from the group consisting of bromide, chloride, iodide, acetate, benzoate, citrate, lactate, malate, nitrate, phosphate, diphosphate, succinate, sulfate, tartrate and tosylate;

provided that when R1 is phenyl, chlorophenyl or benzyl, R2 is phenyl or benzothienyl and R4 is phenyl or aralkyl, then R3 is selected from the group consisting of alkyl, alkenyl and alkynyl; wherein the double bond of the alkenyl or the triple bond of the alkynyl group is at least one carbon atom removed from the point of attachment;

provided further that when R1 is benzyl or methylphenyl, R2 is phenyl or methylphenyl and R4 is methylphenyl or 4-methoxyphenyl, then R3 is selected from the group consisting of alkyl, alkenyl and alkynyl; wherein the double bond of the alkenyl or the triple bond of the alkynyl group is at least one carbon atom removed from the point of attachment;

provided further that when R1 is phenyl, R2 is phenyl and R4 is phenyl, then R3 is selected from the group consisting of C3-8alkyl, alkenyl and alkynyl; wherein the double bond of the alkenyl or the triple bond of the alkynyl group is at least one carbon atom removed from the point of attachment;

and pharmaceutically acceptable salts thereof.

Examples of compounds of Formula I include but are not limited to: 2-(2-methoxyphenyl)-3-(2-methoxyphenyl)-5-phenylamino-[1,2,4]-thiadiazol-2-ium; 2-(2-methoxyphenyl)-3-(2-methoxyphenyl)-5-(2-methoxyphenylamino)-[1,2,4]-thiadiazol-2-ium; 2-(2-methoxyphenyl)-3-(2-methoxyphenyl)-5-(4-tolylamino)-[1,2,4]-thiadiazol-2-ium; 2-(2-methoxyphenyl)-3-phenyl-5-(4-methoxyphenylamino)-[1,2,4]-thiadiazol-2-ium; 2-(2-methoxyphenyl)-3-phenyl-5-(4-tolylamino)-[1,2,4]-thiadiazol-2-ium; 2-(2-methoxyphenyl)-3-phenyl-5-(2-tolylamino)-[1,2,4]-thiadiazol-2-ium; and pharmaceutically acceptable salts thereof.

Examples of compounds of Formula II include but are not limited to [2-(2-methoxyphenyl)-3-(2-methoxyphenyl)-2H-[1,2,4]-thiadiazol-5-ylidene]-phenylamine and pharmaceutically acceptable salts thereof.

The structure of the related thiourea derivatives of 1,2,4-thiadiazole is provided below.

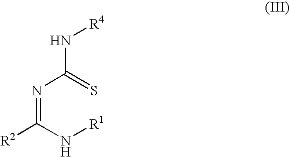

wherein the R groups are as described above for Formula (I)

The preferred thiourea derivative has the formula of:

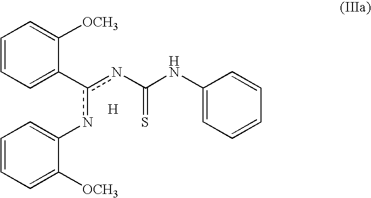

and pharmaceutically acceptable salt thereof.

In one embodiment, the stable composition of the present invention comprises Compound Ia which is 2-(2-methoxyphenyl)-3-(2-methoxyphenyl)-5-phenylamino-[1,2,4]-thiadiazol-2-ium, and pharmaceutically acceptable salts; and the bromide salt, also known as 5-phenylamino-2,3-bis(2-methoxyphenyl)-1,2,4 thiadiazonium, is preferred. The structure of the preferred bromide salt of Compound Ia is provided below.

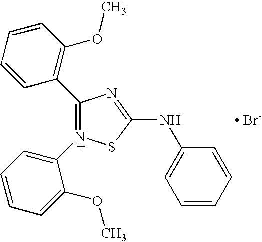

In another embodiment, the stable composition of the present invention comprises Compound IIa which is [2-(2-methoxyphenyl)-3-(2-methoxyphenyl)-2H-[1,2,4]-thiadiazol-5-ylidene]-phenylamine and also known as N-[2,3 -Bis-(2-methoxyphenyl)-1,2,4-thiazol-5(2H)-ylidene]-benzenamine. The structure of Compound IIa is shown below.

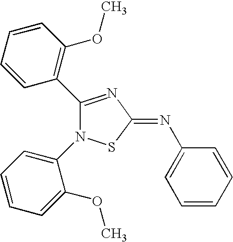

In another embodiment, the stable composition of the present invention comprises Compound IIIb which is 1-[(2-methoxy-phenyl)-(2-methoxy-phenylamino)-methylene]-3-phenyl-thiourea, and pharmaceutically acceptable salt thereof. The structure of Compound IIIb is provided below.

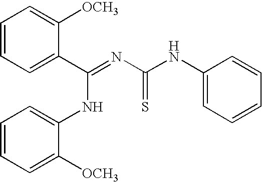

In yet another embodiment, the stable composition of the present invention comprises Compound IIIc, a corresponding tautomeric form of Compound IIIb, which is 1-[(2-methoxy-phenyl)-(2-methoxy-phenylimino)-methyl]-3-phenyl-thiourea, and a pharmaceutically acceptable salt thereof. The structure of Compound IIIc is provided below.

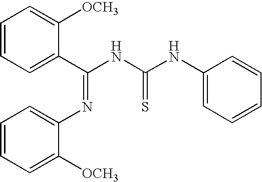

It is found herein that Compounds Ia, IIa, and IIIa have similar properties and are exchangeable in the stable compositions of the present invention. The compound may be present in the compositions in any desired concentration or range. The range of Compound Ia, IIa, and IIIa may be from about 0.05% to about 20% (w/w), preferably from about 0.01% to about 10% (w/w), more preferably from about 0.1% to about 8% (w/w) and most preferably from about 0.6% to about 4% (w/w). The appropriate amount of compound may be varied depending on the severity of the disease and the condition of the patient in need of topical administration.

The stable compositions or formulations provided within the present invention may be present in the form of creams or emulsions, lotions, gels, suspensions, aerosols, foams, and the like. Preferably, the stable formulation or composition is present in aqueous gel, lotion, cream or emulsion, foam, and the like.

The compositions of the present invention have desired and improved properties, including the extended shelf-life or stability and the suitable commercial production. As used herein, the terms ‘stable composition’, ‘stable formulation’, ‘composition with extended shelf-life’ or variants thereof refer to the compound of Formula I, II, or III (and most preferably Compounds Ia, IIa, and IIIa) in the composition is stable. The stability or shelf-life of the compound may be determined by measuring the concentration and any degradation product of the compound of Formula I, II or III in the compositions using high performance liquid chromatography (HPLC). As commonly referred in the art, the compound is considered to be stable or being stabilized when the concentration of the compound retains at least about 90% to at least about 110%, preferably at least about 95% to at least about 105%, of the original concentration of the compound.

By way of example, the stability of the compound of Formula I, II, or III is determined using HPLC equipped with a temperature-controlled autosampler, a thermostatic column compartment, and an UV detector at about 326 nm (Agilent 1100, Waters alliance HPLC). A Zorbax C18 column is used for analyzing Compound Ia and a YMC-Pack Hydrosphere C18 column is used for analyzing Compound IIa. A binary linear gradient of mobile phases A and B is used. The mobile phase A may consist of about 0.2% trifluroacetic acid in water and the mobile phase B may consist of about 0.2% trifluroacetic acid in methanol. The gradient conditions are provided below.

| Time (minutes) | Mobile Phase A (%) | Mobile Phase B (%) |
|---|---|---|
| 0 | 55 | 45 |
| 10 | 40 | 60 |
| 20 | 10 | 90 |
| 25 | 10 | 90 |
| 27 | 55 | 45 |
| 35 | 55 | 45 |

The stable composition according to the present invention has stability or shelf-life of at least six months. Preferably, the aqueous gel of the present invention has stability or shelf-life of at least twelve months, preferably eighteen months and most preferably twenty-four months, under ambient condition of about 25° C. In addition, the cream or emulsion of the present invention has stability or shelf-life of at least six months, preferably twelve months and most preferably eighteen months, under refrigerated condition of about 5° C. To prepare the stable compositions, one or more compounds of Formula I, II and/or III or salt thereof is provided as the active ingredient and mixed with pharmaceutical excipients according to conventional pharmaceutical compounding techniques.

The stable formulations may contain, in addition to the active ingredient(s), one or more non-active components including, but are not limited to chelating agents, buffering agents, pH modifying agents, colorants, preservatives, fragrances, emulsifiers, surfactants, opacifying agents, emollients, solvents, sunscreens, viscosity modifying agents, antioxidants, moisturizers, permeations enhancers, film forming polymers and the like.

In one embodiment, the stable composition is an aqueous gel comprising a compound of Formula I, II, and/or III (preferably Compounds Ia, IIa, and IIIa), a viscosity modifying agent, a preservative and water (Table 1). Optionally, the aqueous gel may include a pH modifying agent, a surfactant, and a film forming polymer.

TABLE 1
Components and ranges for an aqueous gel.

| | Range | Preferred Range |
|---|---|---|
| Viscosity | 10,000-300,000 cps | 50,000-150,000 cps |
| PH | 3.0-7.0 | 4.5-6.0 |
| Compound Ia, IIa, or IIIa | 0.05-20% | 0.1-8% |
| Viscosity modifying agent | 0.1-20% | 0.2-10% |
| Preservative | 0.05-5% | 0.5-1.5% |
| pH modifying agent¹ | 0.05-0.5% | 0.1-0.25% |
| Surfactant¹ | 0.05-2.0% | 0.2-0.8% |
| Film forming polymer¹ | 0.1-5% | 0.2-2% |
| Water | q.s.* | q.s.* |

¹Optionally present.
*Wherein the total weight of all components adds up to 100.

Suitable viscosity modifying agents include but are not limited to acacia, agar, alginic acid, bentonite, carbomer including carbomer copolymer, carbomer homopolymer, and carbomer interpolymer, carboxymethylcellulose calcium, carboxymethylcellulose sodium, carboxymethylcellulose, carrageenan, microcrystalline cellulose and carboxymethylcellulose sodium mixture, dextrin, gelatin, gellan gum, guar gum, hydroxyethyl cellulose, hydroxypropyl cellulose, hydroxypropyl methylcellulose, maltodextrin, methylcellulose, pectin, polyethylene oxide, polyvinyl alcohol, povidone, propylene glycol alginate, pullulan, hydrophobic colloidal silica, silicon dioxide, sodium alginate, corn starch, xanthan gum, and PVM/MA Decadiene Crosspolymer (such as under trade names of STABILEZE™, ULTRATHIX™, LUBRAJEL™, and GANTREZ™). Preferred are pharmaceutical grades of high molecular weight (e.g. in the billions), crosslinked, polyacrylic acid polymers such as Carbomer 974P are preferred to provide the preferred viscosity for the aqueous gel. The range of the viscosity may be from about 10,000 to about 300,000 cps, and preferably from about 50,000 to about 150,000 cps. The viscosity modifying agent may be present in the range of about 0.1% to about 25%, and preferably about 0.2% to about 10% to provide the desired viscosity modifying agent. When carbomer is used (preferably Carbomer 974P) to provide the desired viscosity, it may be present in the range between about 0.1% to about 5% and preferably about 0.5% to about 1.5% by weight.

The viscosity may be determined by any known method, including Brookfield Synchro-Lectric Viscometer (LVT, with helipath stand). By way of example, viscosity measurements are conducted using Spindle F at about 3 rpm at about 25° C.

Suitable preservatives include but are not limited to benzalkonium chloride, benzethonium chloride, benzoic acid, benzyl alcohol, butylparaben, cetrimonium bromide, cetylpyridinium chloride, chlorobutanol, chlorocresol, cresol, dehydroacetic acid, ethylparaben, methylparaben, phenol, phenoxyethanol, phenylethyl alcohol, phenylmercuric acetate, phenylmercuric nitrate, potassium benzoate, potassium sorbate, propylparaben, sodium benzoate, sodium dehydroacetate, sodium propionate, sorbic acid, diazolidinyl urea, imidazolidinyl urea and quaternium-15. Phenoxyethanol is preferred for the aqueous gel. When phenoxyethanol is used, it may be present in the range between about 0.05% to about 5% and preferably about 0.5% to about 1.5% by weight.

Optionally, the pH of the aqueous gel may be adjusted to between about 3.0 and about 7.0, and preferable from about 4.5 to about 6.0. Any pH modifying agent compatible with the compound of Formula I, II, or III or Compounds Ia, IIa, or IIIa may be used. Suitable pH modifying agents include but are not limited to diethanolamine, trolamine, monoethanolamine, sodium hydroxide, tromethamine, triethanolamine and potassium hydroxide. Sodium hydroxide is preferred for the aqueous gel. When sodium hydroxide is used, it may be present in the range of about 0.05% to about 0.5% and preferably from about 0.1% to about 0.25% by weight.

The preferred aqueous gel may comprise Compound Ia, IIa or IIIa, phenoxyethanol, carbomer, sodium hydroxide and water as provided in Table 2.

TABLE 2
Components and ranges for the preferred aqueous gel.

| Component | Range (%, w/w) | Preferred Range (%, w/w) |
|---|---|---|
| Compound Ia, IIa, or IIIa | 0.05-20 | 0.1-8 |
| Phenoxyethanol | 0.5-2 | 0.7-1.5 |
| Carbomer 974P | 0.5-2 | 0.7-1.5 |
| 10% NaOH Solution | 1-4 | 1.5-2.5 |
| Water | q.s.* | q.s.* |

*Wherein the total weight of all components adds up to 100.

In another embodiment of the present invention, a base composition is provided. The base composition is suitable for mixing with the aqueous gel described above in Table 2. The base composition may comprise a preservative, a solvent or a mixture of solvents, a viscosity modifying agent and water (Table 3). Optionally, emulsifiers, chelating agents, pH modifying agents, colorants, fragrances, surfactants, opacifying agents, emollients, sunscreens, antioxidants, moisturizers, permeations enhancers, film forming polymers and the like may be included in the base composition.

TABLE 3
Components and ranges of a base composition.

| | Range | Preferred Range |
|---|---|---|
| Viscosity | 10,000-300,000 cps | 50,000-150,000 cps |
| PH | 3.0-7.0 | 4.5-6.0 |
| Emulsifier¹ | 0.2-10% | 0.5-5% |
| Solvent or solvent mixture | 1-20% | 5-15% |
| Viscosity modifying agent | 0.5-8% | 1.5-5% |
| Preservatives | 0.05-5% | 0.5-1.5% |
| pH modifying agent¹ | 0.05-0.5 | 0.1-0.25 |
| Water | q.s.* | q.s.* |

¹Optionally present.
*Wherein the total weight of all components adds up to 100.

In a preferred embodiment, the base composition may comprise an emulsifier, a mixture of solvents, a preservative, a viscosity modifying agent, a pH modifying agent and water.

Suitable emulsifiers include ionic and nonionic emulsifiers. The ionic emulsifier may include the sodium and potassium salts of sulfated higher primary aliphatic alcohols, such as sodium caprylyl sulfonate, sodium cetyl sulfate, sodium cetearyl sulfate, sodium decyl sulfate, sodium lauryl sulfate, sodium myristyl sulfate, sodium oleyl sulfate, sodium octyl sulfate, sodium tridecyl sulfate and potassium lauryl sulfate. The nonionic emulsifiers may include polyoxyethylene sorbitan esters (e.g. polysorbate 20 and polysorbate 80), sorbitan esters, polyethylene glycol esters, alkoxylated alcohols such as polyoxyethylene stearyl ether, polyethylene ethers, ceteary alcohol and cetearyl glycoside and the like. Other emulsifier such as diethylene glycol stearates, ethylene glycol stearates, glyceryl distearate, glyceryl monolinoleate, glyceryl monooleate, glyceryl monostearate, lanolin alcohols, lecithin, mono- and di-glycerides, oleyl oleate, palm kernel oil, poloxamer, polyoxyethylene 50 stearate, polyoxyl 10 oleyl ether, polyoxyl 20 cetostearyl ether, polyoxyl 35 castor oil, polyoxyl 40 hydrogenated castor oil, polyoxyl 40 stearate, polyoxyl lauryl ether, polyoxyl stearyl ether, polysorbate 20, polysorbate 40, polysorbate 60, polysorbate 80, propylene glycol dicaprylate/dicaprate, propylene glycol monocaprylate, propylene glycol monostearate, superglycerinated fully hydrogenated rapeseed oil, sodium cetostearyl sulfate, sodium lauryl sulfate, sodium stearate, sorbitan monolaurate, sorbitan monooleate, sorbitan monopalmitate, sorbitan monostearate, sorbitan sesquioleate, sorbitan trioleate, stearic acid, and emulsifying wax may also be used. Emulsifying wax such as POLAWAX® (Croda Chemical, UK) is the preferred emulsifier for the base composition. When emulsifying wax is used, it may be present in the range between about 0.2% to about 10% and preferably about 0.5% to about 5% by weight.

Suitable solvents include but are not limited to ethanol, glycol, almond oil, benzyl alcohol, benzyl benzoate, caster oil, corn oil, cottonseed oil, ethyl acetate, ethyl oleate, glycerin, glycofurol, isopropyl alcohol, isopropyl myristate, light mineral oil, medium chain triglycerides, mineral oil, monoethanolamine, olive oil, peanut oil, polyethylene glycol, polyoxyl 35 caster oil, propylene carbonate, propylene glycol, sesame oil, soybean oil, sunflower oil, triacetin, triethanolamine, diethylene glycol monoethyl ether, hexylene glycol, polyethylene glycol monomethyl ether, caprylocaproyl polyoxylglycerides, butyl alcohol, hydrogenated polydecene, lauroyl polyoxylglycerides, linoleoyl polyoxylglycerides, oleoyl polyoxylglycerides and stearoyl polyoxylglycerides; and isopropyl myristate and/or ethanol is preferred. Suitable glycols include but not limited to propylene glycol, diethylene glycol, triethylene glycol, butylenes glycol, hexylene glycol and polyethylene glycol; and propylene glycol is preferred. When isopropyl myristate, ethanol and/or propylene glycol is used, it may be present in the range between about 1% to about 20% and preferably about 5% to about 15%.

Preservatives and viscosity modifying agents suitable for the aqueous gel may be also used for the base composition. Phenoxyethanol is the preferred preservative and carbomer is the preferred viscosity modifying agent for the base composition.

The preferred base composition may comprise emulsifying wax (e.g. POLAWAX®), ethanol, propylene glycol, isopropyl myristate, phenoxyethanol, carbomer, sodium hydroxide and water as provided in Table 4.

TABLE 4

| Component | Range (%, w/w) | Preferred Range (%, w/w) |
|---|---|---|
| POLAWAX ® | 5-15 | 8-10 |
| Ethanol | 2-15 | 7-10 |
| Propylene Glycol | 2-15 | 7-10 |
| Isopropyl Myristate | 2-10 | 4-6 |
| Phenoxyethanol | 0.1-5 | 0.5-2 |
| Carbomer 974P | 0.01-1 | 0.05-0.1 |
| 10% NaOH Solution | 0.1-2 | 0.2-0.6 |
| Water | q.s.* g | q.s.* |

*Wherein the total weight of all components adds up to 100.

In a further embodiment, the stable composition may comprise a mixture of the aqueous gel (preferably as shown in Table 2) and the base composition (preferably as shown in Table 4) in a weight ratio which ranges from about 1:9 to about 9:1 (w/w), preferably about 1:4 to about 4:1 (w/w), more preferably from about 1:2 to about 2:1 (w/w) and most preferably about 1:1 (w/w). The aqueous gel and the base composition may be mixed using any suitable method known in the art, for example a Kitchen-aid mixer or a Hobart mixer.

The preferred composition prepared by a mixture of the aqueous gel and the base composition may comprise the compound of Formula I, II or III, POLAWAX®, ethanol, propylene glycol, isopropyl myristate, phenoxyethanol, Carbomer 974P, sodium hydroxide and water as provided in Table 5.

TABLE 5
Components and ranges of the preferred composition prepared by
mixing the aqueous gel and the base composition.

| Component | Range (%, w/w) | Preferred Range (%, w/w) |
|---|---|---|
| Compound Ia, IIa, or IIIa | −0.1-10 | −0.6-4 |
| POLAWAX ® | 2-10 | 4-6 |
| Ethanol | 2-8 | 3-5 |
| Propylene Glycol | 2-8 | 3-5 |
| Isopropyl Myristate | 1-5 | 2-4 |
| Phenoxyethanol | 0.1-5 | 0.5-2 |
| Carbomer 974P | 0.1-2 | 0.4-0.8 |
| 10% NaOH Solution | 0.5-5 | 1-2 |
| Water | q.s.* | q.s.* |

*Wherein the total weight of all components adds up to 100.

By way of example, the aqueous gel may be packaged in one container and the base composition may be packaged in a separate container. The pharmacist or healthcare professional may mix appropriate amounts of the aqueous gel and the base composition and dispense the mixture for patients or users.

Alternatively, the aqueous gel and the base composition may be packaged in a dual chamber device. The first container in the dual chamber device may contain the aqueous gel and the second container in the dual chamber device may contain the base composition. A user or a patient may dispense and mix the composition from the dual chamber device instantly prior to treatment. The dual chamber device is applicable for either single or multiple uses, depending on its design.

Alternatively, the stable composition may be prepared in a conventional emulsion manufacturing procedure well known in the art, such as the method described in Example 3.

In yet another embodiment, the stable composition is an emulsion comprising a compound of Formula I, II, or III, a viscosity modifying agent, an emulsifier, a solvent or a mixture of solvents, a preservative, a pH modifying agent, and water (Table 6). Optionally, hydrophobic vehicle, chelating agents, antioxidants, buffering agents, colorants, surfactants, emollients, permeation enhancers, film forming polymers, and the like may be included.

TABLE 6
Component and range of an emulsion.

| | Range | Preferred Range |
|---|---|---|
| Viscosity | 10,000-300,000 cps | 50,000-150,000 cps |
| PH | 3.0-7.0 | 4.5-6.0 |
| Compound Ia, IIa, or IIIa | 0.05-10% | 0.6-4% |
| Emulsifier | 0.2-10% | 0.5-5% |
| Solvent or solvent mixture | 1-20% | 5-15% |
| Viscosity modifying agent¹ | 0.5-8% | 1.5-5% |
| Preservatives | 0.05-5% | 0.5-1.5% |
| pH modifying agent | 0.05-0.5% | 0.1-0.25% |
| Water | q.s.* | q.s.* |

¹Optionally present.
*Wherein the total weight of all components adds up to 100.

The preferred emulsion may comprise the compound of Formula I, II, or III, propylene glycol, phenoxyethanol, carbomer, disodium EDTA, emulsifying wax, isopropyl myristate, ethanol, sodium hydroxide, and water as provided in Table 7.

TABLE 7
Components and ranges of the preferred emulsion.

| Component | Range (%, w/w) | Preferred Range (%, w/w) |
|---|---|---|
| Ethanol | 2-10 | 4-5 |
| Propylene Glycol | 2-6 | 3-5 |
| Phenoxyethanol | 1 g | 1 g |
| Carbomer 974P | 0.1-1 | 0.3-0.7 |
| Disodium EDTA | 0.001-0.1 | 0.005-0.5 |
| Emulsifying Wax | 2-8 | 3-6 |
| Isopropyl Myristate | 1-5 | 3-4 |
| Compound Ia | 0.05-10 | 0.6-4 |
| 10% NaOH Solution | 0.5-2 | 1-1.5 |
| Water | q.s.* | q.s.* |

*Wherein the total weight of all components adds up to 100.

In one aspect, the stable compositions may be used to treat any dermatological disease or disorder mediated by a melanocortin receptor, particularly for treatment of acne or extensive sebum production. The stable topical compositions provided herein will contain an amount of the pharmaceutical active ingredient necessary to deliver an effective dose. The dosages may be varied depending upon the requirement of the patients, the severity of the condition being treated and the compound being employed. The use of either daily or other periodic administration may be employed. Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

In another aspect, the present invention provides a method of mixing two compositions, prior to dispensing to the patients in need of treatment, by a pharmacist or a healthcare professional. The first composition comprises the compound of Formula I, II or III in any of the aforementioned stable compositions which is stable at ambient condition. The second composition comprises a base composition which is capable of dispersing or dissolving, completely or partially, the compound of Formula I or II. The first and second compositions may be mixed and dispensed using any suitable method known in the art. By way of example, the first composition may be the aqueous gel packaged in a container and the second composition may be the base composition packaged into a separate container. The pharmacist or healthcare professional may mix appropriate amounts of the aqueous gel and the base composition to form a mixture or cream or emulsion and dispense the mixture to patients or users. The method may further comprise the step of refrigerating the resulting composition for administration of multiple uses.

The kit may further include an instruction to use or a label instructing the user to refrigerate the cream or emulsion following dispensing.

The following Examples are set forth to aid in the understanding of the invention, and are not intended, and should not be construed to limit in any way the invention set forth in the claims which follow thereafter.

### Example 1

### Aqueous Gel

### Aqueous Gel Composition A

TABLE 8
Components of aqueous gel composition A.

| Component | Wt/100 g product |
|---|---|
| Compound Ia | 0.3 g |
| Phenoxyethanol | 1 g |
| Carbomer 974P | 1 g |
| 10% NaOH Solution | 2 g |
| Water | q.s. |

Preparation: In a mixing bowl, water and phenoxyethanol were mixed using a Lightening mixer until phenoxyethanol was dissolved. Carbomer was slowly added and mixed until uniformly dispersed. Compound Ia was added into the mixture and mixed with a spatula. The mixture was transferred to another bowl to a Silverson L4R mixer and mixed until Compound Ia was uniformly dispersed. The mixing bowl was transferred to a Kitchen Aid Mixer and a NaOH solution was added into the mixture while mixing. The mixture was mixed until a homogenous gel was formed.

The stability of Compound Ia was examined by measuring the concentration of Compound Ia using HPLC. The concentration of the compound in the composition stored at various temperatures for a period of time was compared to the initial concentration of the compound prior to storage. The results showed that Compound Ia in the aqueous gel composition A was stable at ambient condition.

### Aqueous Gel Composition B

TABLE 9
Components of aqueous gel composition B.

| Component | Wt/100 g product |
|---|---|
| Compound IIa | 2.40 |
| Phenoxyethanol | 1.00 |
| Carbomer 974P | 1.00 |
| 10% NaOH Solution | 2.00 |
| Water | q.s. |

Preparation: In a mixing bowl, water and phenoxyethanol were mixed using a Lightening mixture until phenoxyethanol was dissolved. Carbomer was slowly added and mixed until uniformly dispersed. Compound IIa was added into the mixture and mixed with a spatula. The mixture was transferred to another bowl and mixed using a Silverson L4R mixer until Compound IIa was uniformly dispersed. The mixing bowl was transferred to a Kitchen Aid Mixer and added with NaOH solution while mixing. Mixed until a homogenous gel was formed.

The same aqueous gel composition B with various concentration 0.3%, 0.6%, 1.2%, 1.5% and 2.4% of Compound IIa was prepared as described above. These aqueous gels were examined for stability as described above and the results were summarized in Table 10. Similar to the results of Compound Ia in the aqueous gel composition A, various amounts of Compound IIa in the aqueous gel composition B were also stable at ambient condition (Table 2).

TABLE 10
Stability of Compound IIa in the aqueous gel composition B.

| | Storage | % of Initial Concentration | | | | |
|---|---|---|---|---|---|---|
| Storage Time | Temperature | 0.3% | 0.6% | 1.2% | 1.5% | 2.4% |
| 12 months | 5° C. | 99.47 | NA | NA | 100.68 | NA |
| | 25° C. | 95.60 | 93.90 | 95.49 | 99.03 | 101.30 |
| 24 months | 5° C. | 97.92 | 97.02 | 97.56 | NA* | NA |
| | 25° C. | 89.49 | 92.69 | 93.87 | NA | NA |

*NA = not assayed

### Example 2

### Composition for Dual Chamber Device

To evaluate a stable composition suitable for a dual chamber device, a base composition and an aqueous gel were prepared separately. The aqueous gel with 2.4% of Compound IIa was prepared as described for composition B in Example 1.

### Base Composition C

TABLE 11
Components of the base composition C.

| Component | Wt/100 g product | |
|---|---|---|
| POLAWAX ® | 9.6 | g |
| Ethanol | 8 | g |
| Propylene Glycol | 8 | g |
| Isopropyl Myristate | 5 | g |
| Phenoxyethanol | 1 | g |
| Carbomer 974P | 0.1 | g |
| 10% NaOH Solution | 0.4 | g |
| Water | 67.9 | g |

Preparation: The base composition was prepared by mixing water, phenoxyethanol, and propylene glycol in a container using a Lightning mixer. Carbomer was slowly added and mixed until uniformly dispersed. The mixture was heated to about 65° C. to about 75° C. and mixed until uniformly dispersed to form a water-phase mixture. In a separate container, emulsifying wax and isopropyl myristate were added, heated to about 65° C. to about 75° C. and mixed using a Lighting mixer to form an oil-phase mixture. The oil-phase mixture was added to the water-phase mixture while mixing using a Lighting mixer. The resulting mixture was mixed and cooled to about 40° C. When cooled to about 40° C., ethanol and 10% NaOH solution were added. The mixture was mixed using a Lightening mixture and cooled to about 30° C. to form the base composition C.

The base composition C and the aqueous gel composition B were mixed at a weight ratio of 1:1 using a Kitchen Aid mixer to obtain a final composition. The ingredients for the resulting composition was provided below in Table 12.

### Cream Composition D

TABLE 12
Components of the cream composition D.

| Component | Wt/100 g product | |
|---|---|---|
| Compound IIa | ~1.2 | g |
| POLAWAX ® | ~4.8 | g |
| Ethanol | ~4 | g |
| Propylene Glycol | ~4 | g |
| Isopropyl Myristate | ~2.5 | g |
| Phenoxyethanol | ~1 | g |
| Carbomer 974P | ~0.55 | g |
| 10% NaOH Solution | ~1.2 | g |
| Water | ~80.75 | g |

~means about.

The stability was examined as Example 1 and the results for the cream composition were summarized in Table 13. The results showed that Compound IIa was stable in the cream composition C at ambient condition for at least 6 months and at refrigerated condition for at least 18 months (Tables 10 and 13).

TABLE 13
Stability of 1.2% Compound IIa in cream composition D.

| Storage Time | Storage Temperature | % of Initial Concentration |
|---|---|---|
| 6 months | 5° C. | 100.0 |
| | 25° C. | 94.1 |
| 12 months | 5° C. | 98.3 |
| 18 months | 5° C. | 100.8 |

### Example 3

### Emulsion

### Composition E

TABLE 14
Components for emulsion composition E.

| Ingredient | Wt/100 g product | |
|---|---|---|
| Water Phase | | |
| Water | ~77.30 | g |
| Propylene Glycol | ~4 | g |
| Phenoxyethanol | ~1 | g |
| Carbomer 974P | ~0.55 | g |
| Disodium EDTA | ~0.01 | g |
| Oil Phase | | |
| Emulsifying Wax | ~4.8 | g |
| Isopropyl Myristate | ~2.5 | g |
| Active Pharmaceutical Ingredient slurry | | |
| Compound IIa | ~1.2 | g |
| Water | ~5.0 | g |
| Ethanol 190 Proof | ~4.4 | g |
| 10% NaOH Solution | ~1.2 | g |

~means about.

Preparation: In a container, water, propylene glycol, phenoxyethanol, disodium EDTA and carbomer 974P were mixed using a Lighting mixer to form a water phase. The water phase was heated to about 65° C. to 75° C. In a separate container, emulsifying wax and isopropyl myristate were mixed using a Lighting mixer to form an oil phase. The oil phase was heated to about 65° C. to 75° C. In another container, water was mixed with Compound IIa to form an active pharmaceutical ingredient slurry. The oil phase was slowly added into the water phase to form an emulsion. When cooled to about 40° C., the active pharmaceutical ingredient slurry and ethanol were slowly added into the emulsion. When cooled to about 30° C., 10% NaOH solution was slowly added to form the composition E.

The stability was examined as Example 1. The results summarized in Table 15 showed that Compound IIa in the emulsion composition E was stable for at least 13 months under refrigerated condition.

TABLE 15
Stability for Compound IIa in the emulsion composition E.

| Storage Time | Storage Temperature | % of Initial Concentration |
|---|---|---|
| 3 months | 5° C. | 101.40 |
| | 25° C. | 98.01 |
| 6 months | 5° C. | 100.11 |
| | 25° C. | 95.61 |
| 9 months | 5° C. | 100.20 |
| 13 months | 5° C. | 100.54 |

### Composition F

TABLE 16
Components of the emulsion composition F.

| Component | Wt/100 g product | |
|---|---|---|
| Water Phase | | |
| Water | 76.36 | g |
| Disodium EDTA | 0.05 | g |
| Xanthan Gum | 0.3 | g |
| Oil Phase | | |
| Cetearyl Alcohol and Cetearyl Glucoside | 7 | g |
| C12-15 Alkyl Benzoate | 8 | g |
| Active Pharmaceutical Ingredient slurry | | |
| Compound Ia | 0.3 | g |
| Isopropyl Myristate | 2 | g |
| Solution A | | |
| Imidazolidinyl Urea | 0.3 | g |
| Citric Acid Monohydrate | 0.42 | g |
| Sodium Phosphate Dibasic Heptahydrate | 0.27 | g |
| Water | 5.0 | g |

Preparation: In a container, water, disodium EDTA and Xanthan gum were mixed using a Lighting mixer to form a water phase. The water phase was heated to about 65° C. to 75° C. In a separate container, cetearyl alcohol and cetearyl glucoside and C12-15 alkyl benzoate were mixed using a Lighting mixer to form an oil phase. The oil phase was heated to about 80° C. to about 85° C. In another container, isopropyl myristate was mixed with Compound Ia to form an active pharmaceutical ingredient slurry. In a separate container, imidazolidinyl urea, citric acid monohydrate and sodium phosphate dibasic heptahydrate were mixed with water to form a solution A. The oil phase was slowly added into the water phase to form an emulsion. When the emulsion was cooled to about 40° C., the solution A was added and mixed. When cooled the emulsion to about 30° C. to 35° C., the active pharmaceutical ingredient slurry was added to form the emulsion composition F.

The stability was examined as Example 1. The results showed that Compound Ia in the emulsion composition F was stable for at least 4 weeks under refrigerated condition.

### Composition G

TABLE 17
Components of the emulsion composition G.

| Component | Wt/100 g product | |
|---|---|---|
| Water Phase | | |
| Water | 66.36 | g |
| Disodium EDTA | 0.05 | g |
| Xanthan Gum | 0.3 | g |
| Oil Phase | | |
| C12-15 Alkyl Benzoate | 8 | g |
| Cetyl Alcohol | 4 | g |
| Stearyl Alcohol | 3 | g |
| Polyoxyethylene (20) Stearyl Ether | 4 | g |
| Polyoxyethylene (2) Stearyl Ether | 1 | g |
| Active Pharmaceutical Ingredient slurry | | |
| Compound Ia | 0.3 | g |
| Isopropyl Myristate | 2 | g |
| Solution A | | |
| Imidazolidinyl Urea | 0.3 | g |
| Citric Acid Monohydrate | 0.42 | g |
| Sodium Phosphate Dibasic Heptahydrate | 0.27 | g |
| Water | 10 | g |

### Composition H

TABLE 18
Components for the emulsion composition H.

| Component | Wt/100 g product | |
|---|---|---|
| Water Phase | | |
| Water | 66.21 | g |
| Disodium EDTA | 0.05 | g |
| Xanthan Gum | 0.3 | g |
| Oil Phase | | |
| Cetyl Alcohol | 4 | g |
| Stearyl Alcohol | 3 | g |
| C12-15 Alkyl Benzoate | 8 | g |
| Polyoxyethylene (20) Stearyl Ether | 4 | g |
| Polyoxyethylene (2) Stearyl Ether | 1 | g |
| Active Pharmaceutical Ingredient slurry | | |
| Compound IIa | 0.1 | g |
| Isopropyl Myristate | 2 | g |
| Solution A | | |
| Imidazolidinyl Urea | 0.3 | g |
| Citric Acid Monohydrate | 0.20 | g |
| Sodium Phosphate Dibasic Heptahydrate | 0.84 | g |
| Water | 10 | g |

Preparation for compositions G and H: In a container, water, disodium EDTA and Xanthan gum were mixed using a Lighting mixer to form a water phase. The water phase was heated to about 65° C. to about 75° C. In a separate container, 12-15 alkyl benzoate, stearyl alcohol, cetyl alcohol, polyoxyethylene (20) stearyl ether and polyoxyethylene (2) stearyl ether were mixed using a Lighting mixer to form an oil phase. The oil phase was heated to about 65° C. to about 75° C. In another container, the isopropyl myristate was mixed with Compound Ia or IIa to form an active pharmaceutical ingredient slurry. In separate container, imidazolidinyl urea, citric acid monohydrate, and sodium phosphate dibasic heptahydrate and water were mixed using a Lightning mixer to form a solution A. The oil phase was slowly added into the water phase to form an emulsion. While cooled to about 40° C., the solution A to the emulsion. When cooled the emulsion to about 30° C. to 35° C., the active pharmaceutical ingredient slurry was added.

The stability was examined as Example 1. The results showed that Compound Ia in the emulsion G and Compound IIa in the emulsion composition H were stable at refrigerated condition for at least 4 weeks.

### Example 4

### Skin Permeation Study

In vitro skin permeation studies were conducted to evaluate the penetration of Compound IIa at various concentrations in an aqueous gel B and the cream composition D.

The cadaver skin finite dose technique was used for the studies. Split thickness human cadaver trunk skin was obtained from skin bank and stored in a water-impermeable plastic bag at about −70° C. Prior to each experiment, skin was thawed in water bath at about 37° C., then cut into sections large enough to fit on the Franz diffusion cell. The diffusion cells were mounted in a diffusion apparatus and filled with a receptor solution of ethanol/water/formic acid (25/75/0.3), which was stirred at about 600 RPM and maintained at about 32° C. The skin was mounted onto the diffusion cell, where the stratum corneum side faced the donor compartment and the dermal side faced the receiving compartment. About 10 μl per cm² of the composition was applied to the skin using a pipette. The dose was spread throughout the skin surface with a Teflon tip of the pipette. After about 4, 8, 12 and 24 hrs, the receptor solution was removed and replaced with fresh receptor solution. An aliquot was taken for analysis. The skin surface was washed with about 0.5 ml of a solvent mixture of methanol/water/formic acid (90/10/0.3). The solvent mixture was filtered and an aliquot was taken for analysis. Subsequently, the dermis and epidermis were separated and Compound IIa was extracted using the solvent mixture. The solvent mixture was filtered and an aliquot was taken for analysis. Samples were analyzed using HPLC.

As summarized in Tables 19 and 20, Compound IIa in both cream composition D and aqueous gel formulation B was able to penetrate into epidermis, dermis and receptor solution. Also, the results showed that the total penetration amount in epidermis, dermis and receptor solution increased as the concentration of Compound IIa increased in the formulations.

TABLE 19
Total amount (ug) of Compound IIa permeated from the cream
composition E into different skin layers and the receptor solution.

Concentrations of
Compound IIa (% w/w)

| | 0.30% | 0.60% | 1.2% | 2.4% |
|---|---|---|---|---|
| Epidermis | 0.279 | 0.357 | 0.428 | 0.681 |
| Dermis | 0.003 | 0.015 | 0.014 | 0.028 |
| Receptor | 0.015 | 0.025 | 0.025 | 0.070 |

TABLE 20
Total amount (ug) of Compound IIa permeated from the aqueous gel
formulation B into different skin layers and the receptor solution.

| | Concentrations of | | |
|---|---|---|---|
| | Compound IIa (% w/w) | | |
| | 0.30% | 0.60% | 1.2% |
| Epidermis | 0.361 | 0.164 | 0.537 |
| Dermis | 0.004 | 0.007 | 0.007 |
| Receptor | 0.020 | 0.045 | 0.074 |

What is claimed is:

1. A topical composition comprising a compound of Formula I, II or III in an amount of about 0.05% to about 20% by weight, a viscosity modifying agent in an amount of about 0.1% to about 5% by weight, a preservative in an amount of about 0.05% to about 5% by weight, and water added in an amount quantum sufficiat to provide a total of 100% by weight;
wherein said compound of Formula I, II or III has the structure of 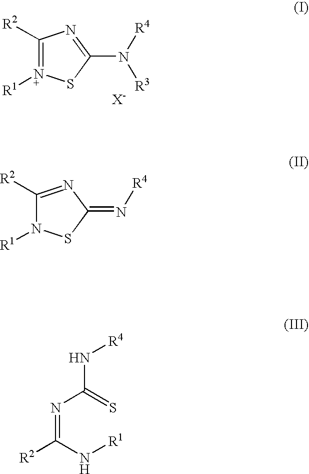
wherein:
R1 is selected from the group consisting of aryl, aralkyl, heteroaryl, heteroaryl-alkyl, heterocycloalkyl, heterocycloalkyl-alkyl, cycloalkyl and cycloalkyl-alkyl; wherein the aryl, aralkyl, heteroaryl, heterocycloalkyl, heterocycloalkyl-alkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;
R2 is selected from the group consisting of aryl, aralkyl, heteroaryl, heterocycloalkyl and cycloalkyl-alkyl; wherein the aryl, aralkyl, heteroaryl, heterocycloalkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;
R3 is selected from the group consisting of hydrogen, alkyl, alkenyl and alkynyl; wherein the double bond of the alkenyl or the triple bond of the alkynyl group is at least one carbon atom removed from the point of attachment;
R4 is selected from the group consisting of aryl, aralkyl, heteroaryl, heterocycloalkyl, and cycloalkyl-alkyl; wherein the aryl, aralkyl, heteroaryl, heterocycloalkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;
X— is selected from the group consisting of bromide, chloride, iodide, acetate, benzoate, citrate, lactate, malate, nitrate, phosphate, diphosphate, succinate, sulfate, tartrate and tosylate;
provided that when R1 is phenyl, chlorophenyl or benzyl, R2 is phenyl or benzothienyl and R4 is phenyl or aralkyl, then R3 is selected from the group consisting of alkyl, alkenyl and alkynyl; wherein the double bond of the alkenyl or the triple bond of the alkynyl group is at least one carbon atom removed from the point of attachment;
provided further that when R1 is benzyl or methylphenyl, R2 is phenyl or methylphenyl and R4 is methylphenyl or 4-methoxyphenyl, then R3 is selected from the group consisting of alkyl, alkenyl and alkynyl; wherein the double bond of the alkenyl or the triple bond of the alkynyl group is at least one carbon atom removed from the point of attachment;
provided further that when R1 is phenyl, R2 is phenyl and R4 is phenyl, then R3 is selected from the group consisting of C3-8alkyl, alkenyl and alkynyl; wherein the double bond of the alkenyl or the triple bond of the alkynyl group is at least one carbon atom removed from the point of attachment;
and pharmaceutically acceptable salts thereof.

2. The topical composition of claim 1, wherein said compound is selected from the group consisting of 2-(2-methoxyphenyl)-3-(2-methoxyphenyl)-5-phenylamino-[1,2,4]-thiadiazol-2-ium, [2-(2-methoxyphenyl)-3-(2-methoxyphenyl)-2H-[1,2,4]-thiadiazol-5-ylidene]-phenylamine, 1-[(2-methoxy-phenyl)-(2-methoxy-phenylamino)-methylene]-3-phenyl-thiourea, 1 -[(2-methoxy-phenyl)-(2-methoxy-phenylimino)-methyl]-3-phenyl-thiourea; and pharmaceutically acceptable salts thereof.

3. The topical composition of claim 2, wherein said compound is [2-(2-methoxyphenyl)-3-(2-methoxyphenyl)-2H-[1,2,4]-thiadiazol-5-ylidene]-phenylamine.

4. The topical composition of claim 1, wherein said viscosity modifying agent is selected from the group consisting of acacia, agar, alginic acid, bentonite, carbomer copolymer, carbomer homopolymer, and carbomer interpolymer, carboxymethylcellulose calcium, carboxymethylcellulose sodium, carboxymethylcellulose, carrageenan, microcrystalline cellulose and carboxymethylcellulose sodium , dextrin, gelatin, gellan gum, guar gum, hydroxyethyl cellulose, hydroxypropyl cellulose, hydroxypropyl methylcellulose, maltodextrin, methylcellulose, pectin, polyethylene oxide, polyvinyl alcohol, povidone, propylene glycol alginate, pullulan, hydrophobic colloidal silica, silicon dioxide, sodium alginate, corn starch, and xanthan gum.

5. The topical composition of claim 4, wherein said viscosity modifying agent is selected from the group consisting of carbomer copolymer, carbomer homopolymer, and carbomer interpolymer.

6. The topical composition of claim 1, wherein said preservative is selected from the group consisting of benzalkonium chloride, benzethonium chloride, benzoic acid, benzyl alcohol, butylparaben, cetrimonium bromide, cetylpyridinium chloride, chlorobutanol, chlorocresol, cresol, dehydroacetic acid, ethylparaben, methylparaben, phenol, phenoxyethanol, phenylethyl alcohol, phenylmercuric acetate, phenylmercuric nitrate, potassium benzoate, potassium sorbate, propylparaben, sodium benzoate, sodium dehydroacetate, sodium propionate, sorbic acid, diazolidinyl urea, imidazolidinyl urea and quaternium-15.

7. The topical composition of claim 6, wherein said preservative is phenoxyethanol.

8. The topical composition of claim 1, further comprising a surfactant in an amount of about 0.05% to about 2% by weight.

9. The topical composition of claim 1, further comprising a film forming polymer in an amount of about 0.1% to about 5% by weight.

10. The topical composition of claim 1, further comprising a pH modifying agent in an amount of about 0.05% to about 0.5% by weight.

11. The topical composition of claim 10, wherein said compound is in an amount of about 0.6% to about 4% by weight, said viscosity modifying agents in an amount of about 0.7% to about 1.5% by weight, said preservative is in an amount of about 0.7% to about 1.5% by weight, said pH modifying agent is in an amount of about 0.1% to about 0.25% by weight, and water in an amount quantum sufficiat to provide a total of 100% by weight.

12. The topical composition of claim 11, wherein said compound is [2-(2-methoxyphenyl)-3-(2-methoxyphenyl)-2H-[1,2,4]-thiadiazol-5-ylidene]-phenylamine, said viscosity modifying agent is Carbomer 974P, said preservative is phenoxyethanol, and said pH modifying agent is sodium hydroxide.

13. The topical composition of claim 1, further comprising a mixture of solvents in an amount of about 1% to about 20% by weight, an emulsifier in an amount of about 0.2% to about 10% by weight, and a pH modifying agent in an amount of about 0.05% to about 0.5% by weight.

14. The topical composition of claim 13, wherein said compound is selected from the group consisting of 2-(2-methoxyphenyl)-3-(2-methoxyphenyl)-5-phenylamino-[1,2,4]-thiadiazol-2-ium, [2-(2-methoxyphenyl)-3-(2-methoxyphenyl)-2H-[1,2,4]-thiadiazol-5-ylidene]-phenylamine, 1-[(2-methoxy-phenyl)-(2-methoxy-phenylamino)-methylene]-3-phenyl-thiourea, 1-[(2-methoxy-phenyl)-(2-methoxy-phenylimino)-methyl]-3-phenyl-thiourea; and pharmaceutically acceptable salts thereof.

15. The topical composition of claim 14, wherein said compound is [2-(2-methoxyphenyl)-3-(2-methoxyphenyl)-2H-[1,2,4]-thiadiazol-5-ylidene]-phenylamine.

16. The topical composition of claim 13, wherein said solvent is selected from the group consisting of ethanol, isopropyl alcohol, benzyl alcohol, butyl alcohol, propylene glycol, diethylene glycol, triethylene glycol, butylenes glycol, hexylene glycol, polyethylene glycol, almond oil, benzyl alcohol, benzyl benzoate, caster oil, corn oil, cottonseed oil, ethyl acetate, ethyl oleate, glycerin, glycofurol, isopropyl alcohol, isopropyl myristate, light mineral oil, medium chain triglycerides, mineral oil, monoethanolamine, olive oil, peanut oil, polyethylene glycol, polyoxyl 35 caster oil, propylene carbonate, propylene glycol, sesame oil, soybean oil, sunflower oil, triacetin, triethanolamine, diethylene glycol monoethyl ether, hexylene glycol, polyethylene glycol monomethyl ether, caprylocaproyl polyoxylglycerides, butyl alcohol, hydrogenated polydecene, lauroyl polyoxylglycerides, linoleoyl polyoxylglycerides, oleoyl polyoxylglycerides and stearoyl polyoxylglycerides.

17. The topical composition of claim 16, wherein said solvent is selected from the group consisting of isopropyl myristate, ethanol, and propylene glycol.

18. The topical composition of claim 13, wherein said emulsifier is selected from the group consisting of sodium caprylyl sulfonate, sodium cetyl sulfate, sodium cetearyl sulfate, sodium decyl sulfate, sodium lauryl sulfate, sodium myristyl sulfate, sodium oleyl sulfate, sodium octyl sulfate, sodium tridecyl sulfate, potassium lauryl sulfate, polyoxyethylene sorbitan esters, sorbitan esters, polyethylene glycol esters, polyoxyethylene stearyl ether, polyethylene ethers, ceteary alcohol, cetearyl glycoside, diethylene glycol stearates, ethylene glycol stearates, glyceryl distearate, glyceryl monolinoleate, glyceryl monooleate, glyceryl monostearate, lanolin alcohols, lecithin, mono- and di-glycerides, oleyl oleate, palm kernel oil, poloxamer, polyoxyethylene 50 stearate, polyoxyl 10 oleyl ether, polyoxyl 20 cetostearyl ether, polyoxyl 35 castor oil, polyoxyl 40 hydrogenated castor oil, polyoxyl 40 stearate, polyoxyl lauryl ether, polyoxyl stearyl ether, polysorbate 20, polysorbate 40, polysorbate 60, polysorbate 80, propylene glycol dicaprylate/dicaprate, propylene glycol monocaprylate, propylene glycol monostearate, superglycerinated fully hydrogenated rapeseed oil, sodium cetostearyl sulfate, sodium lauryl sulfate, sodium stearate, sorbitan monolaurate, sorbitan monooleate, sorbitan monopalmitate, sorbitan monostearate, sorbitan sesquioleate, sorbitan trioleate, stearic acid, and emulsifying wax.

19. The topical composition of claim 18, wherein said emulsifier is POLAWAX®.

20. The topical composition of claim 13, wherein said compound is [2-(2-methoxyphenyl)-3-(2-methoxyphenyl)-2H-[1,2,4]-thiadiazol-5-ylidene]-phenylamine, said viscosity modifying agent is Carbomer 974P, said preservative is phenoxyethanol, said solvent is selected from the group consisting of isopropyl myristate, ethanol, and propylene glycol, said emulsifier is POLAWAX®, and said pH modifying agent is sodium hydroxide.

21. The topical composition of claim 20, wherein [2-(2-methoxyphenyl)-3-(2-methoxyphenyl)-2H-[1,2,4]-thiadiazol-5-ylidene]-phenylamine is in an amount of about 0.6% to about 4% by weight, POLAWAX® is in an amount of about 4% to about 6% by weight, ethanol is in an amount of about 3% to about 5% by weight, propylene glycol is in an amount of about 3% to about 5% by weight, isopropyl myristate is in an amount of about 2% to about 4% by weight, phenoxyethanol is in an amount of about 0.5% to about 2% by weight, Carbomer 974P is in an amount of about 0.4% to about 0.8% by weight, sodium hydroxide is in an amount of about 0.1% to 0.25% by weight, and water is in an amount quantum sufficiat to provide a total of 100% by weight.

22. A kit for a topical composition comprising:
a) a first container comprises an aqueous gel comprising a compound of Formula I, II or III in an amount of about 0.05% to about 20% by weight, a viscosity modifying agent in an amount of about 0.1% to about 5% by weight, a preservative in an amount of about 0.05% to about 5% by weight, and water in an amount quantum sufficiat to provide a total of 100% by weight;
wherein said compound of Formula I, II or III has the structure of 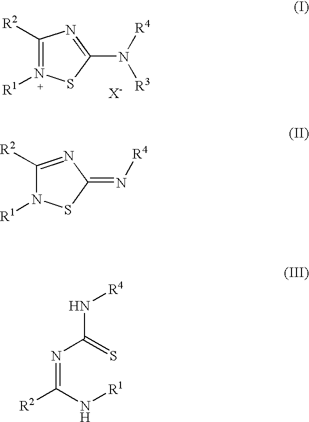
wherein:
R1 is selected from the group consisting of aryl, aralkyl, heteroaryl, heteroaryl-alkyl, heterocycloalkyl, heterocycloalkyl-alkyl, cycloalkyl and cycloalkyl-alkyl; wherein the aryl, aralkyl, heteroaryl, heterocycloalkyl, heterocycloalkyl-alkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;
R2 is selected from the group consisting of aryl, aralkyl, heteroaryl, heterocycloalkyl and cycloalkyl-alkyl; wherein the aryl, aralkyl, heteroaryl, heterocycloalkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;
R3 is selected from the group consisting of hydrogen, alkyl, alkenyl and alkynyl;
wherein the double bond of the alkenyl or the triple bond of the alkynyl group is at least one carbon atom removed from the point of attachment;
R4 is selected from the group consisting of aryl, aralkyl, heteroaryl, heterocycloalkyl, and cycloalkyl-alkyl; wherein the aryl, aralkyl, heteroaryl, heterocycloalkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;
X— is selected from the group consisting of bromide, chloride, iodide, acetate, benzoate, citrate, lactate, malate, nitrate, phosphate, diphosphate, succinate, sulfate, tartrate and tosylate;
provided that when R1 is phenyl, chlorophenyl or benzyl, R2 is phenyl or benzothienyl and R4 is phenyl or aralkyl, then R3 is selected from the group consisting of alkyl, alkenyl and alkynyl; wherein the double bond of the alkenyl or the triple bond of the alkynyl group is at least one carbon atom removed from the point of attachment;
provided further that when R1 is benzyl or methylphenyl, R2 is phenyl or methylphenyl and R4 is methylphenyl or 4-methoxyphenyl, then R3 is selected from the group consisting of alkyl, alkenyl and alkynyl; wherein the double bond of the alkenyl or the triple bond of the alkynyl group is at least one carbon atom removed from the point of attachment;
provided further that when R1 is phenyl, R2 is phenyl and R4 is phenyl, then R3 is selected from the group consisting of C3-8alkyl, alkenyl and alkynyl; wherein the double bond of the alkenyl or the triple bond of the alkynyl group is at least one carbon atom removed from the point of attachment;
and pharmaceutically acceptable salts thereof; and
b) a second container comprises a base comprising a mixture of solvents in an amount of about 1% to about 20% by weight, a viscosity modifying agent in an amount of about 0.5% to about 8% by weight, a preservative in an amount of about 0.05% to about 5% by weight; an emulsifier in an amount of about 0.2% to about 10% by weight, a pH modifying agent in an amount of about 0.05% to about 0.5% by weight, and water in an amount quantum sufficiat to 100% by weight.

23. A method of preparing a topical composition comprising mixing:
a) an aqueous gel comprising a compound of Formula I, II or III in an amount of about 0.05% to about 20% by weight, a viscosity modifying agent in an amount of about 0.1% to about 5% by weight, a preservative in an amount of about 0.05% to about 5% by weight, and water in an amount quantum sufficiat to provide a total of 100% by weight;
wherein said compound of Formula I, II or III has the structure of 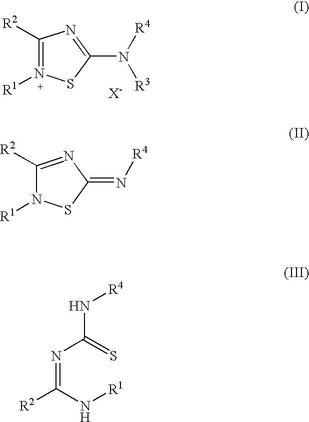
wherein:
R1 is selected from the group consisting of aryl, aralkyl, heteroaryl, heteroaryl-alkyl, heterocycloalkyl, heterocycloalkyl-alkyl, cycloalkyl and cycloalkyl-alkyl; wherein the aryl, aralkyl, heteroaryl, heterocycloalkyl, heterocycloalkyl-alkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;
R2 is selected from the group consisting of aryl, aralkyl, heteroaryl, heterocycloalkyl and cycloalkyl-alkyl; wherein the aryl, aralkyl, heteroaryl, heterocycloalkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;
R3 is selected from the group consisting of hydrogen, alkyl, alkenyl and alkynyl; wherein the double bond of the alkenyl or the triple bond of the alkynyl group is at least one carbon atom removed from the point of attachment;
R4 is selected from the group consisting of aryl, aralkyl, heteroaryl, heterocycloalkyl, and cycloalkyl-alkyl; wherein the aryl, aralkyl, heteroaryl, heterocycloalkyl or cycloalkyl group is optionally substituted with one or more substituents independently selected from halogen, hydroxy, alkyl, alkoxy; halogenated alkyl, halogenated alkoxy, amino, alkylamino or di(alkyl)amino;
X— is selected from the group consisting of bromide, chloride, iodide, acetate, benzoate, citrate, lactate, malate, nitrate, phosphate, diphosphate, succinate, sulfate, tartrate and tosylate;
provided that when R1 is phenyl, chlorophenyl or benzyl, R2 is phenyl or benzothienyl and R4 is phenyl or aralkyl, then R3 is selected from the group consisting of alkyl, alkenyl and alkynyl; wherein the double bond of the alkenyl or the triple bond of the alkynyl group is at least one carbon atom removed from the point of attachment;
provided further that when R1 is benzyl or methylphenyl, R2 is phenyl or methylphenyl and R4 is methylphenyl or 4-methoxyphenyl, then R3 is selected from the group consisting of alkyl, alkenyl and alkynyl; wherein the double bond of the alkenyl or the triple bond of the alkynyl group is at least one carbon atom removed from the point of attachment;
provided further that when R1 is phenyl, R2 is phenyl and R4 is phenyl, then R3 is selected from the group consisting of C3-8alkyl, alkenyl and alkynyl; wherein the double bond of the alkenyl or the triple bond of the alkynyl group is at least one carbon atom removed from the point of attachment; and pharmaceutically acceptable salts thereof; with
b) a base comprising a mixture of solvents in an amount of about 1% to about 20% by weight, a viscosity modifying agent in an amount of about 0.5% to about 8% by weight, a preservative in an amount of about 0.05% to about 5% by weight; an emulsifier in an amount of about 0.2% to about 10% by weight, a pH modifying agent in an amount of about 0.05% to about 0.5% by weight, and water in an amount quantum sufficiat to provide a total of 100% by weight;
to form a substantially homogeneous topical composition.

24. The method of claim 23, wherein said aqueous gel and said base are mixed in a weight ratio of 1:9 to 9:1.

25. The method of claim 23, wherein said aqueous gel and said base are mixed in a weight ratio of 1:4 to 4:1.

26. The method of claim 23, wherein said aqueous gel and said base are mixed in a weight ratio of 1:2 to 2:1.

27. The method of claim 23, wherein said aqueous gel and said base are mixed in a weight ratio of 1:1.

28. A topical composition comprising:
a compound of [2-(2-methoxyphenyl)-3-(2-methoxyphenyl)-2H-[1,2,4]-thiadiazol-5-ylidene]-phenylamine in an amount of about 0.6% to about 4% by weight,
propylene glycol in an amount of about 3% to about 5% by weight,
phenoxyethanol in an amount of about 0.5% to about 1.5% by weight,
carbomer in an amount of about 0.3% to about 0.7% by weight,
disodium EDTA in an amount of about 0.005% to about 0.5% by weight,
emulsifying wax in an amount of about 3% to about 6% by weight,
isopropyl myristate in an amount of about 3% to about 4% by weight,
ethanol in an amount of about 4% to about 5% by weight
sodium hydroxide in an amount of about 0.1% to about 0.25% by weight, and
water in an amount quantum sufficiat to provide a total of 100% by weight.

29. A method of treating a dermatological disease or disorder mediated by a melanocortin receptor in a subject in need thereof comprising administering an effective amount of said topical composition of claim 28.

30. The method of claim 29, wherein said disease is acne.

31. The method of claim 29, wherein said disorder is extensive sebum production.
